(19) United States
(12) Patent Application Publication
(10) Pub. No.: US 2011/0098559 A1
(43) Pub. Date: Apr. 28, 2011

(54) Guiding insert assembly for a catheter used with a catheter position guidance system

(76) Inventors: William John Besz, South Australia (AU); Donald Philip Chorley, South Australia (AU)

(21) Appl. No.: 11/795,281

(22) PCT Filed: Jan. 12, 2006
(86) PCT No.: PCT/AU06/00027

§ 371 Date: Jun. 29, 2009

Related U.S. Application Data
(60) Provisional application No. 60/644,179, filed on Jan. 14, 2005.

(30) Foreign Application Priority Data
Jan. 14, 2005 (AU) 2005900142

(51) Int. Cl. A61B 5/05

(52) U.S. Cl. 600/424

(57) ABSTRACT

A catheter position guidance system, for use in conjunction with a guiding insert assembly, having an electrical connector, an elongated conductor operatively coupled to the connector, an elongated stiffener coupled to the connector and an electromagnetic field radiator including a support device coupled to the elongated stiffener and operatively coupled to the conductor, the electromagnetic field radiator having an inductance-enhancing element operatively coupled thereto.

### CROSS REFERENCE TO RELATED APPLICATION

This application incorporates by reference the following co-pending patent application: “Catheter Locator Apparatus and Method of Use,” filed on 21 Aug. 2003 having Australian Patent Application 2001283703 (83703/01) assigned to Micronix Pty Ltd.

### BACKGROUND OF THE INVENTION

Physicians and other health care providers frequently use catheters to treat patients. An example of a known catheter includes a tube that is inserted into the human body.

Certain catheters are commonly inserted through the patient's nose, mouth or surgical incision to gain access to the gastrointestinal tract. These catheters sometimes referred to as enteral catheters are used to provide nutrients to the patient and are typically referred to as feeding tubes. The distal end of the feeding tube is placed into the stomach or intestines, and a feeding bag delivers liquid nutrient, liquid medicine or a combination of the two through the tube to the patient. Other functions that enteral catheters are used for include gastric decompression and functional motility studies.

Other types of catheters are inserted into the patient's veins or arteries to gain access to the cardiovascular system. These catheters include, among others, central venous catheters, peripheral venous catheter and peripherally inserted central catheter (PICC). These catheters are often multi-lumen tubes that are passed through the patient's veins or arteries. The health care provider uses these catheters for diagnostic purposes and to provide patients with medications, drugs, fluids, nutrients, or blood products over a period of time, typically several days up to several months.

When using any of the abovementioned types of catheters, it is important to place the distal end of the catheter at the preferred site within the human body. Incorrect placement of the catheter tip may endanger the patient or decrease functionality of the catheter. For example, if the health care provider places an enteral catheter into the patient's lungs by mistake, liquid intended for the stomach or bowel may be introduced into the lungs with harmful consequences. If the health care provider places a catheter into the wrong site within the cardiovascular system, the patient may experience complications such as irritation of the vein leading to cardiac tamponade or increased incidence of thrombus formation, which can impair the function of the catheter. Furthermore any medication passing into the tube will be delivered to the incorrect site.

In some cases, health care providers use X-ray machines to gather information that is used to confirm the correct placement of the catheter tip within the body. There are several disadvantages with using X-ray machines. For example, these machines are relatively large and cumbersome to use, require highly trained operators and expose the patient to radiation in instances where other methods might suffice. Also, due to their size, these machines are typically not readily accessible for use because, they are usually installed in a special X-ray room that is not necessarily convenient for any particular patient.

Best practice for radiation hygiene mandates the use of X-rays only in instances where there is a high benefit relative to the inherent risks.

There are also constraints in the effectiveness of X-rays to demonstrate correct placement that are inherent in the technology. Optimal two-dimensional representation of a three dimensional set of objects on X-rays is subject to operator technique and requires expert interpretation.

Therefore, health care providers can find it inconvenient and expensive to use X-ray machines for assistance or confirmation of their catheter placement procedures. Furthermore, X-ray machines are inconvenient to transport to enable delivery of catheter placement procedures at their hospital bedside or at the patient's home for immediate confirmation of the location of the distal end of the catheter.

Accordingly, there is a need to overcome one or more of these disadvantages so as to enable timely treatment of the patient.

Electronic guidance systems are used to assist the health care provider to place the distal end of catheters to a chosen target area. Such electronic guidance systems may utilize the principle of inductive sensing of an electromagnetic signal. The guiding insert described herein may function as a radiator or a sensor for these systems and includes the use of a radiator or sensor located at or near the tip of the catheter being inserted that can be detected and its relative position indicated to the heath care provider during or after insertion of the catheter into the patient. Furthermore, the guiding insert assembly can be used by the health care provider to manipulate the distal end of the catheter through the various passageways and cavities inside the patient, as it is usable to stiffen the catheter along its length by itself or in combination with the catheter to allow the useful manipulation of the catheter.

The electronic guidance systems can be used in conjunction with the guiding insert assembly for catheters and used for the placement of catheters suitable for enteral or parenteral feeding and the other above-mentioned catheter types for their particular uses.

Electronic guidance systems can be used in conjunction with a guiding insert assembly for catheters, and further applications include placement of Endotracheal Tubes, peritoneal dialysis catheters, Epidural, peripheral neurological catheters, investigational catheters and interventional catheters. An electronic guidance system used in conjunction with a guiding insert assembly in the placement of cardiology catheters can also be useful for endoscopic investigation and percutaneous endoscopic gastrectomy catheter location, these being only some of the possible uses of such a system. Without the guiding insert assembly of the type described above, the catheter can be used with stiffening wire sometimes referred to as a stylet or used over a previously inserted guide-wire but typically only in conjunction with traditional location assistance, such as protocols and X-ray guidance and/or X-ray confirmation to locate the distal end of the catheter in the patient.

Accordingly, there is a need to overcome or minimize the disadvantages described and provide a means to better utilize electronic guidance systems when used in conjunction with a guiding insert assembly to locate the distal end of a catheter.

### SUMMARY OF THE INVENTION

The present invention generally relates to a catheter position guidance system. The catheter position guidance system is used to help guide a catheter to a position located within the body. The system can be used, and is not limited to the location of catheters for enteral, parenteral or other suitable catheter feeding applications or for the delivery of drugs to the heart or other parts and organs of the body by other types of catheters. A catheter and guiding insert assembly is used in conjunction with the catheter position guidance system.

The guiding insert assembly may also use a tubing assembly for covering a portion of the guiding insert assembly.

Both the tubing assembly and guiding insert assembly are relatively lightweight so as not to change in any appreciable way the use of a catheter when being positioned, are easy to use and disposable and can be used with the catheter position guidance system in any health care environment and particularly at a patient's bedside or at a patient's home. The catheter itself is also a single use item but remains in the patient for the period of treatment and or diagnosis as required.

The guiding insert assembly may in one embodiment include a connector, an elongated stiffening wire assembly coupled to the connector and a signal conductor wire. An electromagnetic field radiator is formed from the signal conductor wire at its distal end which is located adjacent the distal end of the elongated stiffening wire. The proximal end of the signal conductor wire is connected to the catheter position guidance system via the connector. The elongated stiffening wire assembly is used to stiffen the catheter into which it is placed and used to assist the health care provider in guiding the distal end of the catheter to a desired location in the body. The tubing assembly includes a protective tube surrounding a portion of the guiding insert assembly that lies between the catheter and the connector.

In one embodiment, the electromagnetic field radiator is shaped as a coil and includes inside the coil an inductance-enhancing element both of which are deployed, in use, at the distal end of the catheter. The electromagnetic field radiator may have associated with it an inductance-enhancing element comprising a high magnetic-permeability material, commonly applied as a core. The inductance-enhancing element enhances the electromagnetic field generated by the electromagnetic field radiator. This enables receiver elements of the catheter position guidance system to receive a stronger signal from the electromagnetic field radiator while the guiding insert assembly is located within the body while at the distal end of the catheter, than without an inductance-enhancing element being associated with the electromagnetic field radiator.

In another embodiment, the guiding insert assembly may include a connector, a tube for carrying a signal conductor wire and a connection of the signal conductor wire to a connector. The tube may be of a type that is similar in mechanical characteristics to the catheter in which it is used or it may be stiffer than the catheter in which it is used. The signal conductor wire is located in the tube or by any way incorporated into the tube and an electromagnetic field radiator is formed from the signal conductor wire and located adjacent the distal end of the tube. The electromagnetic field radiator may be incorporated into the tube material or located external to the tube. The electromagnetic field radiator may be in the form of a coil wound about a former. The electromagnetic field radiator may be associated with an inductance-enhancing element comprising a high magnetic-permeability material. The inductance-enhancing element may be the former about which the electromagnetic field radiator is formed.

The guiding insert assembly and optional tubing assembly are used in conjunction with other components of the catheter position guidance system to assist the health care provider in performing a catheter placement procedure. Also, the electromagnetic field radiator, in one embodiment, is associated with an inductance-enhancing element that enhances the performance of the catheter position guidance system. Therefore, the guiding insert assembly and optional tubing assembly, used in conjunction with the catheter position guidance system of the present invention provide an enhancement to catheter placement methods during medical treatment over that which are currently available.

An advantage of the present invention is in the provision of an electromagnetic field radiator for a guiding insert assembly of a catheter position guidance system.

Yet, another advantage of the present invention is to assist the user in properly placing a catheter end within the body.

Still another advantage of the present invention is to reduce the amount of time necessary to properly guide a catheter to a desired cavity within the body.

Yet, another advantage of the present invention is to reduce the amount of radiation exposure associated with machines that assist in catheter placement and confirmation of placement.

Another advantage of the present invention is to reduce the likelihood of harm caused by placing a catheter within the body.

Yet, another advantage is to simplify the process of catheter placement procedures.

Still another advantage of the present invention is to increase the safety of catheter placement procedures.

Yet, another advantage of the present invention is to assist health care providers in guiding and locating catheters within the body at the patient's bedside.

Another advantage of the present invention is to increase the convenience of obtaining catheter placement information during and after placement of a catheter.

Yet, another advantage of the present invention is to increase the functionality of the catheter.

Another advantage of the present invention is to allow the health care provider to place the catheter with guidance at the patient's bedside.

Additional features and advantages of the present invention are described in, and will be apparent from, the following Detailed Description of the Invention and the figures.

### BRIEF DESCRIPTION OF THE FIGURES

FIG. 1 is a perspective view of the catheter position guidance system illustrating the display device, guiding insert assembly and hand-held receiver (part of the position guidance system) being used to position a catheter within a patient in one embodiment of the present invention.

FIG. 2 is schematic block diagram of the electronic configuration of the catheter position guidance system illustrating the processor, memory device, signal generator, input devices and output devices in one embodiment of the present invention.

FIG. 3 is a top or plan view of the guiding insert assembly and the display device illustrating an enteral application involving a catheter inserted into a human body and indication of catheter information on the display device.

FIG. 4 is a top or plan view of the guiding insert assembly and the display device illustrating a parenteral application involving a catheter inserted into a human body and indication of catheter information on the display device.

FIG. 5 is a perspective view of the end member or tip of the catheter in one embodiment of the present invention.

FIG. 6 is a perspective view of the electromagnetic field radiator illustrating the tubular insulator housing a portion of the signal conductor wire assembly and an elongated stiffener in one embodiment of the present invention.

FIG. 7A is a top or plan view of the circuit board of the electrical connector of the electromagnetic field radiator in one embodiment of the present invention.

FIG. 7B is a rear perspective view of the contact block that houses electrical contacts.

FIG. 8 is a perspective view of the electromagnetic field radiator in one embodiment of the present invention suitable for Central Venous Catheter applications.

FIG. 9 is a perspective view of the electromagnetic field radiator illustrating the coils surrounding the inductance-enhancing element in one embodiment of the present invention.

FIG. 10 is a cross-section view of the electromagnetic field radiator and the inductance-enhancing element taken substantially along line XXI-XXI of FIG. 9 illustrating spirals of the outer coil uniformly patterned with spirals of the inner coil surrounding the inductance-enhancing element and the elongated stiffener coupled to the inductance-enhancing element.

FIG. 11 is a side view of a portion of a guiding insert including a signal conductor wire twisted over a stiffening wire.

FIG. 12 is a cross-section view of the guiding insert taken substantially along line A-A.

FIG. 13 is a cross-section view of one embodiment of a guiding insert taken substantially along its longitudinal axis at its distal end.

FIG. 14 is a cross-section view of another embodiment of a guiding insert taken substantially along its longitudinal axis at its distal end.

FIG. 15 is a cross-section view of a guiding insert taken substantially along its longitudinal axis at its distal end when deployed in a PICC type catheter.

FIG. 16 is a catheter and a guiding insert located adjacent each other before the guiding insert is deployed into the catheter.

### DETAILED DESCRIPTION OF THE INVENTION

### I. Catheter Position Guidance System

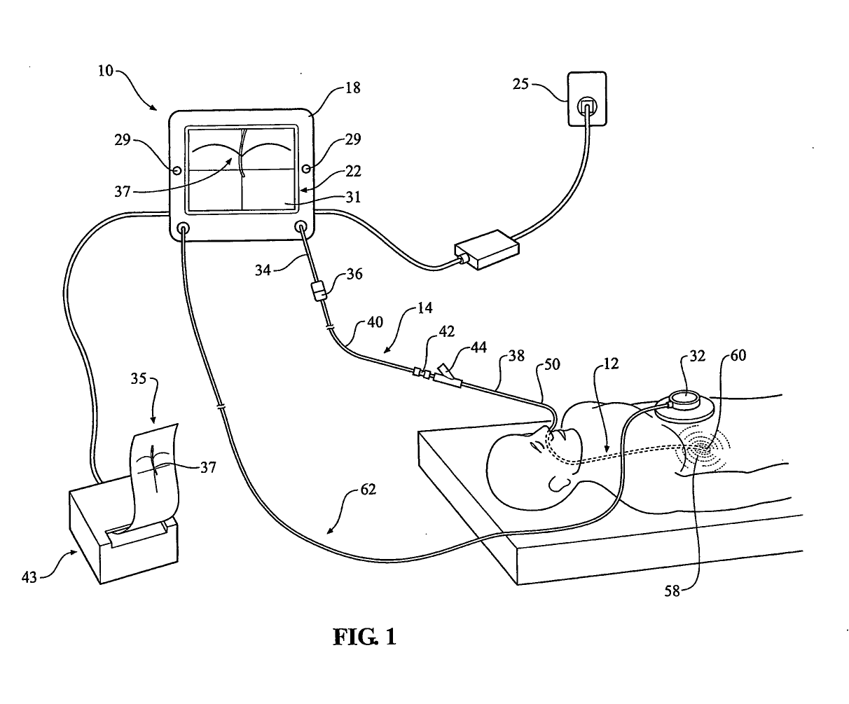

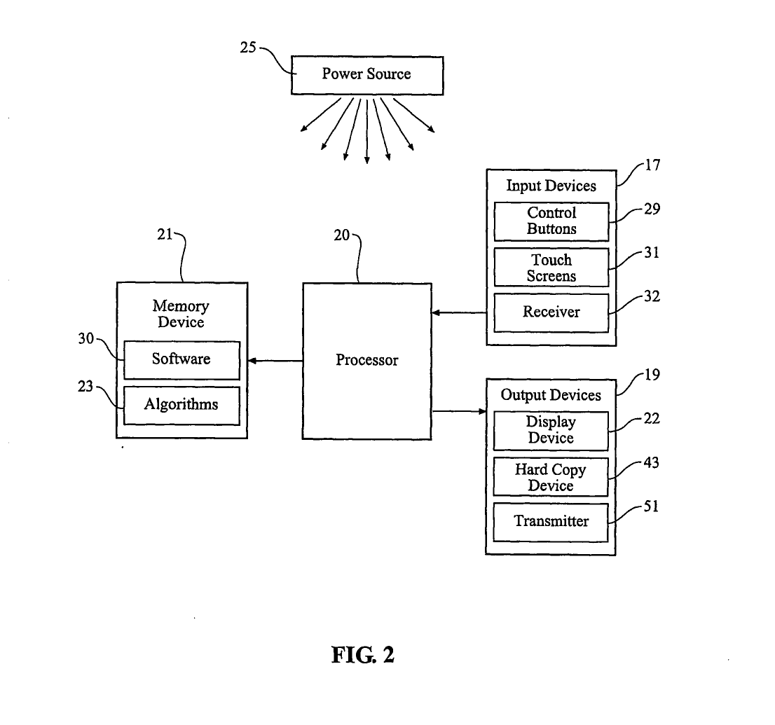

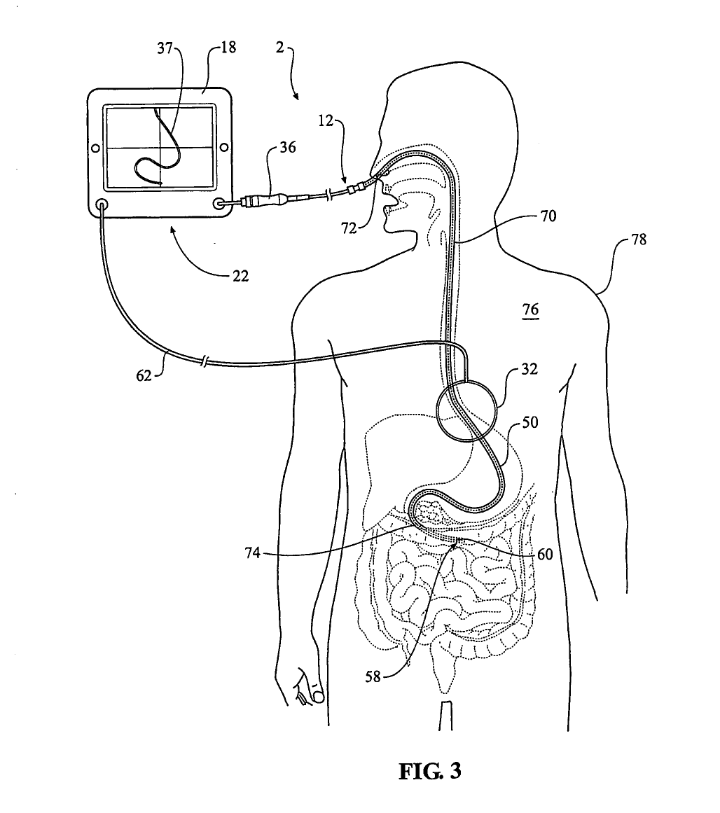

Referring now to the drawings, in an embodiment illustrated in FIGS. 1 and 2, the catheter position guidance system 2 includes: (a) an apparatus 10 having a housing 18 which supports a controller or processor 20 (FIG. 2) and a display device 22; (b) a non-invasive movable electromagnetic signal receiver 32 electrically coupled to the processor 20 by a wire, cable, signal data connection or signal carrier 62; (c) a power source 25; (d) a hard copy device 43 electronically coupled to the apparatus 10 for, in one example, printing out paper or slips 35 having data and/or graphics 37 which indicate relative catheter location information also indicated on the display device 22; and (e) a guiding insert assembly 12 (FIG. 3) in communication with the receiver 32 and operatively coupled to the apparatus 10 by a wire, cable, chord or electrical extension 34, which, in turn, is operatively coupled to the processor 20.

It should be appreciated that the device 32 is a receiver in one embodiment described in detail herein, where an electromagnetic field radiator of the guiding insert assembly, is located inside the body of the patient. However, the device 32 may include a signal receiver and a signal transmitter that operate independently of each other to respectively receive and transmit a signal from and to a respective electromagnetic field radiator or receiver element located inside the body of the patient. Alternatively, the device 32 may include a transmitter to transmit a signal into the body of the patient to be received by a receiver element located therein being part of the guiding insert assembly.

As best illustrated in FIG. 2, the system 2, in one embodiment, includes: (a) a plurality of input devices 17 for providing input signals to the system 2 such as one or more control buttons 29, a touch screen 31 (that may be incorporated into the display device 22 as can the control buttons 29) and the receiver 32; (b) an electromagnetic field radiator 58 which radiates signals that are received by the receiver 32; (c) a memory device 21 including machine readable instructions and one or more computer programs (which, for example, may include a software program 30 and a plurality of algorithms 23) which are used by the processor 20 to process the signal data transmitted by the electromagnetic field radiator 58 and received by the receiver 32, as well as processing the type of guiding insert assembly being used and the various control buttons operated or touch screen instructions required to operate the system; and (d) a plurality of output devices 19 such as the display device 22, the hard copy device 43 both of which indicate catheter tracking information to the health care provider and a signal transmitter device 51 for coupling to the electromagnetic field radiator 58. The display device 22 may be any suitable display including, but not limited to, a liquid crystal display (LCD), light-emitting diode (LED) display, cathode-ray tube display (CRT), or plasma screen.

Health care providers can use the system 2 in a variety of catheter positioning applications. In one example illustrated in FIG. 3, the system 2 is used in an enteral application. Here, a portion 70 of the guiding insert assembly 12 is placed through the patient's nose 72 (or mouth). The distal end or tip 60 of the guiding insert assembly 12 is positioned in the most appropriate location for enteral feeding which in FIG. 3 is shown in the jejunum 74, which is accessed by placing the catheter through the pyloric orifice of the stomach to gain internal access to the intestines of the patient. The health care provider places the receiver 32 over the chest area 76 of a body 78 in accordance with procedures associated with the particular catheter position guidance system and the display provides an indication of the path of the distal end of the catheter while it is being placed. In particular, the display device 22 and the hard copy device 43 indicate information related to the location of the portion 60 which is adjacent the electromagnetic field radiator 58 of the guiding insert assembly 12 within the body 78, as well as information related to the shape of the pathway taken by the guiding insert assembly 12 over time. It should be appreciated that the system 2 need not indicate the exact location or path of the guiding insert assembly 12 to assist the clinician in the placement of the distal end of the catheter at a desired location in the patient. In one embodiment, the display device 22 indicates where the distal end of the guiding insert assembly 12 is positioned with respect to an anatomical template of human anatomy in a way that is fully described in the co-pending application referred to herein. The health care provider uses the graphical indications 37 as a guide that is helpful in properly placing the distal end of the guiding insert assembly 12 in the jejunum 74 in preparation for enteral feeding.

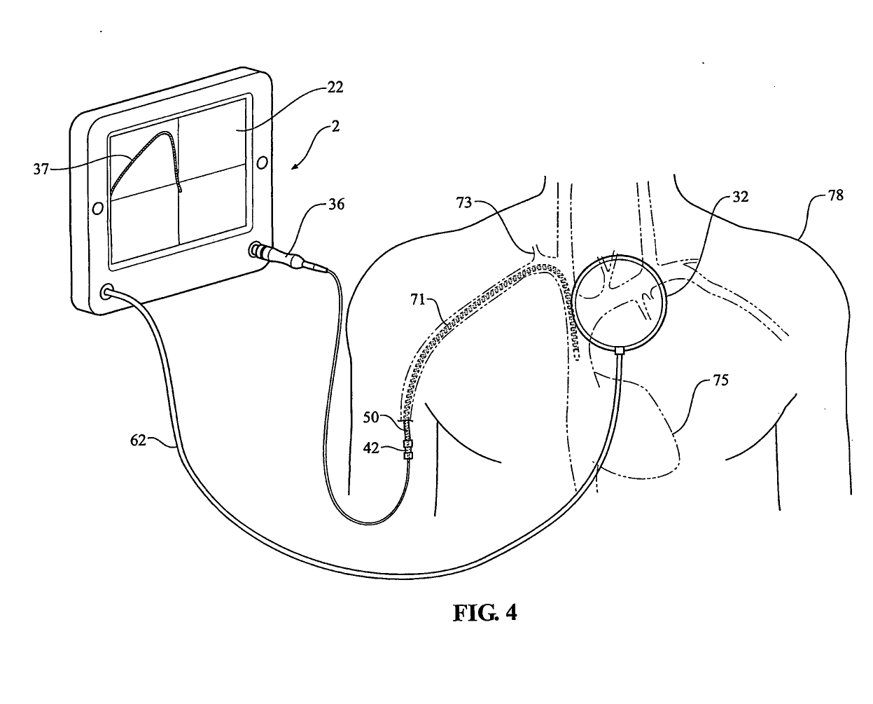

In another example illustrated in FIG. 4, a portion 71 of the guiding insert assembly 12 (which is not necessarily the same configuration as detailed in the earlier figures) is introduced into the patient's body 78 through a vein or artery 73 leading to the heart 75. The system 2 assists the health care provider in guiding the portion 71 of the guiding insert assembly 12 in the patient's vein or artery 73 to a desired cavity in the heart 75 in preparation for drug or other liquid delivery.

### II. Catheter

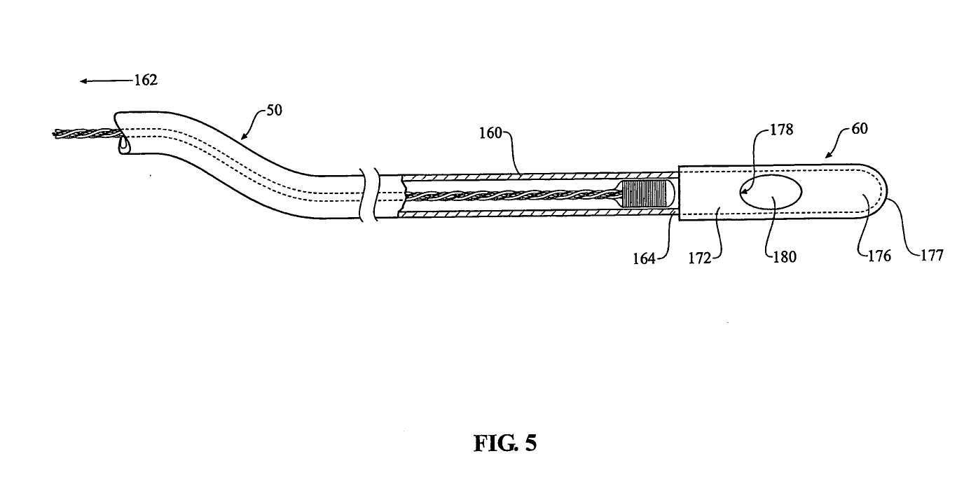

As illustrated in FIG. 5, in one embodiment, an example catheter 50 includes a feeding tube having a body 160 with: (a) a proximal end 162 (not shown); (b) a distal end 164 and (c) an external surface. The proximal end 162 is insertable into a catheter connection branch that can be of the y-port connector type (not shown) to bring the catheter 50 into fluid communication with the y-port connector. In one embodiment, the external surface has a plurality of volumetric, measurement or unit markings uniformly spaced along the body 160 of the catheter 50 (not shown). Markings function as placement markers, which assist the user in assessing the depth that the catheter 50 (feeding tube) is placed within the body.

As best illustrated in FIG. 5, in one embodiment, the end member tip 60 (which may have various configurations to suit the application) of the catheter 50 is attached to the distal end 164 of the catheter. The tip 60 includes a body 172 and an end portion 176. The body 172 defines a passage 178 and an opening 180. The opening 180 is positioned between the collar 174 and the end portion 176. A forward portion 177 of the end portion 176 can have a rounded shape. The shape of the opening 180 of the tip 60 is configured to facilitate the flow of fluid from the catheter 50 into the patient's body while decreasing the likelihood that the opening 180 will become clogged.

The y-port connector (not shown), catheter 50 (feeding tube) and tip 60 (FIG. 1) can each be made from any suitable polymer or plastic material including, but not limited to, polyamide, polyethylene, polypropylene, polyurethane, silicone and polyacrylonitrile. The catheter remains in the body for as long as it is required while the guiding insert assembly including the electromagnetic field radiator is retracted so that the catheter can be used as required.

### III. Guiding Insert Assembly I

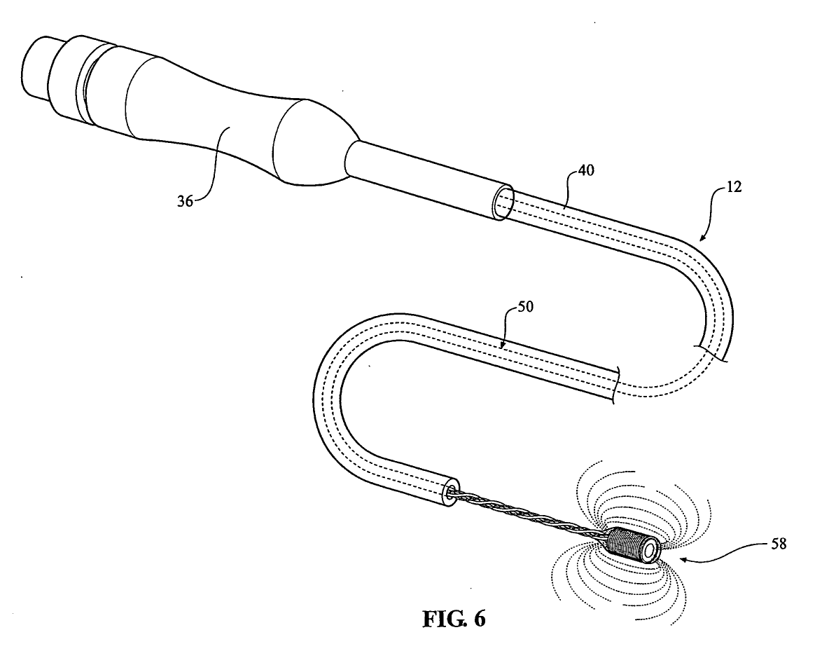

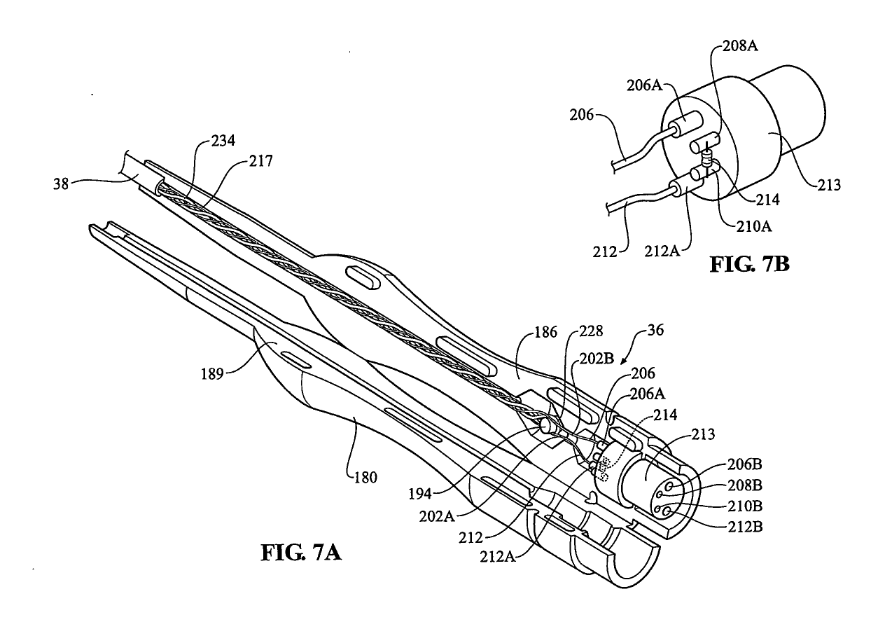

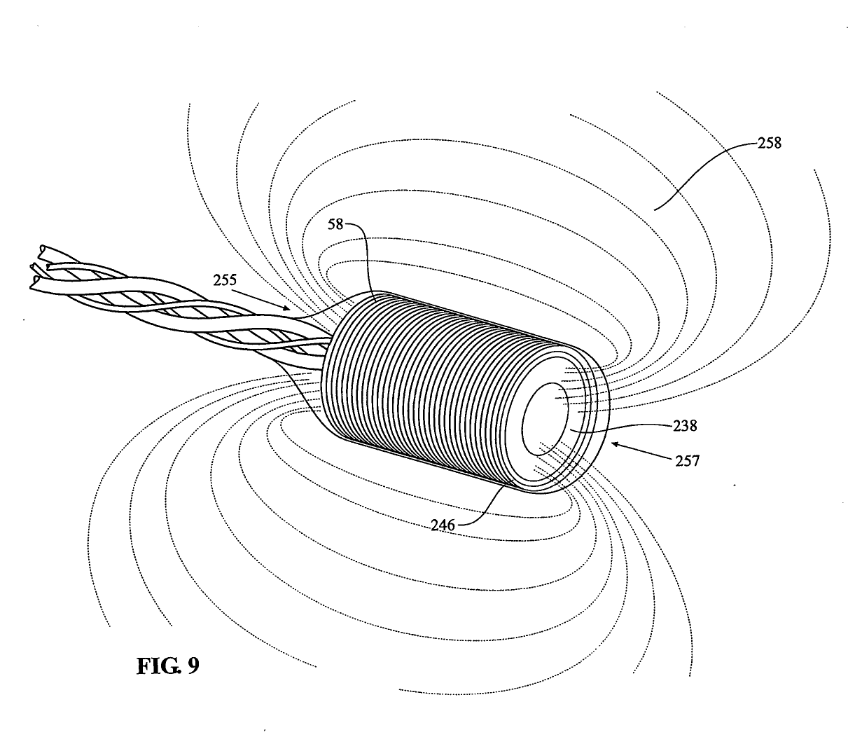

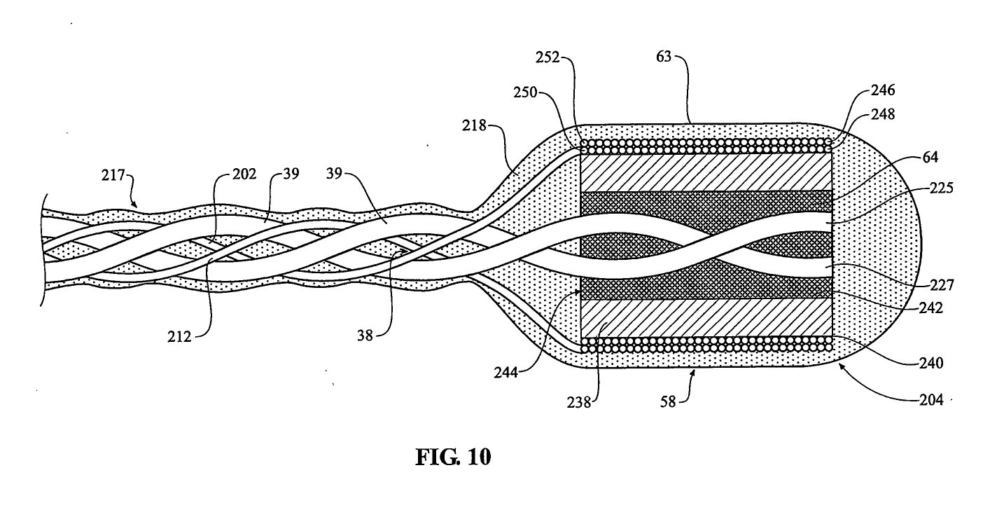

As best illustrated in FIGS. 6-10, in one embodiment most suited to larger internal diameter catheters, the guiding insert assembly 12 assembly includes: (a) an electrical connector 36 operatively connected to the processor 20 (not shown); (b) a signal conductor wire assembly 38 operatively coupled to the connector 36 (FIGS. 7A and 10); (c) an elongated stiffener 39 (this element when used alone is best known as a “stylet” that is used to stiffen the catheter 50 and assist the manipulation of the catheter into position) coupled to the connector 36 and serving in this embodiment as a support for the signal conductor wire assembly 38; (d) an electromagnetic field radiator 58 (FIGS. 6, 8 and 10) operatively part of the signal conductor wire and located at the distal end of the signal conductor wire assembly 38; (e) an inductance-enhancing element 238 (FIGS. 9 and 10), in this embodiment, connected to a distal end 226 of the elongated stiffener 39 and, in this embodiment, serving as a support for the electromagnetic field radiator 58; (f) a protectant 63 (FIG. 10) covering or encapsulating the electromagnetic field radiator 58; and (g) a fastener 64, in this embodiment, attaching the distal end 226 of the elongated stiffener 39 to the electromagnetic field radiator 58 (FIG. 10). The tubular cover 40 (FIG. 6) covers a portion of the signal conductor wire assembly 38 up to, in the embodiment depicted in FIG. 1, the y-connector while the remainder of the guiding insert assembly 12 continues within the catheter 50 (FIG. 6).

It is possible to encapsulate a guiding insert assembly that includes a connector, an elongate stiffener, signal conduction wire and electromagnetic field radiator by covering the assembly with heat shrink material and applying heat to contract it about the assembly parts thus providing a protective cover to the assembly.

Other guiding insert assemblies will be described under the same heading later in the specification.

### IV. Connector

As best illustrated in FIG. 7A, in one embodiment, the connector 36 includes: an electronic lead assembly or electronic connector 180 that is located internally of the body of the connector 36. Although the embodiment illustrated in FIG. 7A has multiple fasteners, it should be understood that, in other embodiments, a single fastener might securely position the block 213 between the two surfaces 186 and 189 (FIG. 7A) that make up the two-part body of the connector 36.

As best illustrated in FIGS. 7A & B and 10, in one embodiment, the signal conductor wire assembly 38 includes: (a) first end 206 of an elongated flexible conductor such as a copper wire 202 (or other suitable low resistance conductor) connected to a terminal post 206A and (b) a second end 212 of the signal conductor wire 202 is connected to the terminal post 212A. Both terminal posts extend longitudinally through the block 213 and provide respective sockets 206B and 212B for connection to another connector or directly to the processor located in the apparatus 10.

The ends 202A and 202B of copper wire can be soldered or otherwise mechanically or chemically connected to the appropriate terminal post 206A and 212A or attached by any suitable means, which may include a crimping arrangement achieved in one embodiment not shown by the closing of the two parts of the body of the connector 36 as long as electrical continuity is maintained between the copper wire and the terminal post. A suitable chemical fastener includes a fastener selected from the group consisting of an adhesive, chemical bond, weld bond and moulding that can be used in other areas of the assembly to fix elements together.

In another embodiment that could replace terminal posts and block 213 a circuit board is provided having contact members formed on a respective circuit board track. The circuit board is arranged to extend from the ends of the signal conduction wire 202 to the proximal end of the body of the connector 36 and having suitable connectors for achieving electrical connection to the processor located in the apparatus 10.

FIG. 7B depicts additional terminal posts 208A and 210A on the block 213 between which can be attached a pre-determined value of resistance in the form of a discrete resistor 214. Both terminal posts extend longitudinally through the block 213 and provide respective sockets 208B and 210B for connection to another connector or directly to the processor located in the apparatus 10. The resistance value, 214 determined by the processor using ancillary electronics and software, will indicate to the processor what type of guiding insert is connected to the processor so that pre-determined factors can be taken account of when processing the signals to be radiated by the electromagnetic field radiator 58 and received by the receiver 32. The amount of resistance measured across those connections is unique to the type of guiding insert assembly to which the connector is fixed. The resistance measurement is made by the catheter position guidance system when the connector is used and thus the system can know automatically which of a variety of guiding inserts is being used at the time.

FIG. 6 depicts a connector 36 and guiding insert assembly 12 having the wire stiffener type at the distal end of which is located an electromagnetic field radiator 58.

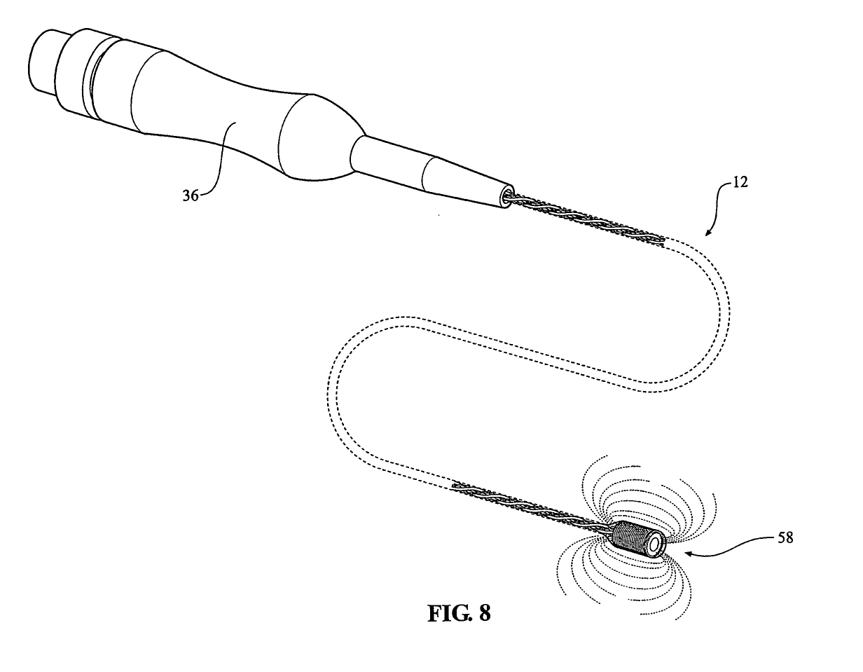

FIG. 8 is a perspective view of the electromagnetic field radiator in one embodiment of the present invention suitable for Central Venous Catheter applications. The connector 36 is attachable directly to the processor via the apparatus 10 and the guiding insert assembly 12 formed from the signal conduction wire 202 having at a distal end of the guiding insert assembly 12 an electromagnetic field radiator 58 with a tubing cover 400 along its length.

The distal end 204 of the signal conductor wire assembly 39 forms the electromagnetic field radiator 58.

In one embodiment depicted in FIG. 10, the distal end of the copper wire 204 is formed into a coil configuration forming the electromagnetic field radiator 58. The copper wire is wound about a former, which in one embodiment, consists of a low permeability material of generally cylindrical shape 238 in FIGS. 9 and 10. The electromagnetic field radiator 58 is formed from a plurality of spirals produced by wrapping a portion of the copper wire around the former that in another embodiment is an inductance-enhancing element 238. The single wire is wound about the former in two layers of carefully aligned coils. Such a method produces a cross-sectional coil shape as depicted in FIG. 10. The coil forms an approximate outer diameter of 0.02 mm and an approximate length of 0.035 mm.

Referring to FIGS. 7A and 10, the signal conductor wire 202 is twisted around in this embodiment, the elongated stiffener 39. The twisting of the signal conductor wire cancels or minimizes the radiation of signal along its length, and is depicted in FIGS. 7A & B and 10 as having a twisted configuration 217 about the elongated stiffener 39. In one embodiment, the signal conductor wire is twisted approximately 500 twists per metre along its length, although a signal conductor wire assembly 38 may include any suitable number of twists with as many as 600 twists per metre being suitable. Accordingly, the hand-held receiver 32 receives less, if any, signal interference arising from any electromagnetic fields generated by a signal conductor wire assembly 38.

### V. Elongated Stiffener

As illustrated in FIG. 7A, although the connector disclosed can be used for any type of guiding insert, in one embodiment, the connector is shown in use with a guiding insert having a wire stiffening element. The elongated stiffener 39 includes a bend at portion 228. The elongated stiffener 39 is preferably made of ferrous material, for example, steel wire but can be made of any other suitable material as it preferably acts like a stylet for assisting the manipulation of the end of the catheter in the patient for the health care provider while placing the catheter into position in the body of the patient. The centre portion 228 of the elongated stiffener 39 is in this embodiment looped around the fastener 194 of the connector 36 and the wire of the stiffener is twisted around itself forming a twisted configuration 234. In another embodiment, the bend at portion 228 would suffice itself to retain the wire in the connector once the two halves were fixed together.

The twisted configuration 234 of the wire increases the rigidity of the elongated stiffener 39. The elongated stiffener terminates adjacent the distal end of the catheter in which it is used and in one embodiment is coupled to a former supporting the electromagnetic field radiator 58. The elongated stiffener described above is formed from a single wire hence the centre portion 228. However, in another embodiment the elongated stiffener may include two separate wires that are twisted together and mechanically but non-conductively coupled at each end to the connector body and electromagnetic field radiator respectively.

### VI. Electromagnetic Field Radiator

As best illustrated in FIGS. 9 and 10, in one embodiment, the electromagnetic field radiator 58 is formed by a plurality of spiral windings of the signal conductor wire. In one embodiment, the apparatus 10 transmits alternating electrical current through the signal conductor wire to cause the radiation of an electromagnetic field, preferably only, radiated from the electromagnetic field radiator 58 that has been, in use, located at the distal end of the catheter 50. Although the embodiment illustrated includes coils as the electromagnetic field radiator 58, it should be appreciated that the electromagnetic field radiator 58 can include any alternate suitable mechanism or device, which generates or produces electromagnetic energy or an electromagnetic field to create the field.

In one embodiment, the electromagnetic field radiator 58 includes a low magnetic permeability element 238 (FIGS. 9 and 10), such as, for example, a permanent magnet, resistive magnet or super-conducting magnet and may be of the amorphous magnetic material type or any other suitable material.

In one embodiment, the inductance-enhancing element 238 includes a cylindrical element having ferrite characteristics. In one embodiment, such ferrite characteristics include, but are not limited to, a characteristic of any suitable compound of ferrite oxide with another oxide or otherwise any suitably Ferro-magnetic compound having a relatively high magnetic permeability. The inductance-enhancing element 238 increases or adds to the transmission of the electromagnetic flux, energy or field 258 (FIG. 9) from the electromagnetic field radiator 58 to the receiver 32 (not shown). In one embodiment, inductance-enhancing element 238 is an object having a cylindrical shape or in another embodiment a rod shape. When a ferrite element is used as an inductance-enhancing element, the magnetic field produced by the ferrite atoms augments the electromagnetic field produced by the coils 246 and 248, thereby strengthening the signal capable of being received by the receiver 32. It should be appreciated that any suitable number of coils and any suitable alternating current may be used, and that one element of the strength of the electromagnetic field radiated by the electromagnetic field radiator depends on the number of coils and one of the many other factors is the level and frequency of the electrical current.

In one embodiment, best illustrated in FIG. 10, the inductance-enhancing element 238 has a shape including an outer surface 240 and an inner wall 242 defining an opening 244. It should be appreciated that the opening 244 need not extend all the way through the core or inductance-enhancing element 238. The signal conductor wire 202 is wrapped around the outermost surface 240 to form a coil. As described above, the coil is formed from a plurality of spirals.

It is preferable that the spirals have a uniform pattern. In one embodiment, each individual spiral 252 is placed at the same x-axis location as the corresponding spiral underlying spiral 250. In an alternative embodiment, each spiral 252 is placed off-centre over each underlying spiral 250, each of the embodiments forming a uniform pattern of the spirals. The uniform pattern of spirals (particularly the former described pattern) increases the vector sum of the electromagnetic fields produced by the coil. It should be appreciated that any suitable pattern of spirals 250 and 252, which facilitates the radiation of an electromagnetic field from the coil, can be used. The polymeric coating 218 of the signal conductor wire is relatively thin. Consequently, the signal conductor wire can be wrapped around the inductance-enhancing element 238 in relative close proximity to one another, thereby increasing space efficiency. The efficiency of space keeps the outer diameter of the coil to a minimum and in particular, for being small enough to fit inside various catheters or signal conductor wire carriers (about which more will be described later in this specification).

The coating or protectant 63 encapsulates or covers the coil and the inductance-enhancing element 238. This protectant 63 protects the coils 246 and 248 from fluids even though in normal use there is only a small period of time the tubing assembly will be in the catheter 50 in the body. Any liquid proofing substance or liquid retardant suitable for this purpose may be used to cover or encapsulate the coils, including, without limitation, polymers and ultraviolet resistant adhesives. The most distal external surfaces of the surface coating is made smooth as is the general shape of the external surface of the electromagnetic field radiator 58 assembly to minimize or obviate any damage to the internal parts of the body of the patient into which the catheter is located, as the distal end of the guiding insert assembly may at times extend past the distal end of the catheter. This is less likely for catheters made from polymeric materials and is possible for catheters made of silicon.

The fastener 64 attaches portions 225 and 227 of the elongated stiffener 39 inside the inner wall 242 of the inductance-enhancing element 238. Therefore, the user's catheter and guiding insert assembly insertion force is transmitted from the elongated stiffener 39 to the inductance-enhancing element 238 substantially without transferring any force to the signal conduction wire. Any form of non-conductive adhesive, epoxy or other suitable fastener can be used to secure the elongated stiffener 39 to the inner wall 242. Other arrangements of fastening the elongated stiffener to the inductance-enhancing element or the coil could be used in place of or in addition to the fasteners described.

In operation, when the apparatus 10 sends electrical current to the electromagnetic field radiator (a coil in this embodiment), the coil radiates a signal or electromagnetic field 258 capable of being detected by the non-invasive receiver 32. In one example illustrated in FIG. 9, during one cycle of the signal an electromagnetic field 258 flows from a proximal end 255 of the coil, through the air and other materials nearby, and the field re-enters the distal end 257 of the coil when the current flowing in the coil is in an appropriate direction. Within the inductance-enhancing element 238, the field 258 flows from the distal end back to the proximal end of the element. Consequently, when the signal conductor wire takes the form of a coil about the inductance-enhancing element, their shapes create an enhanced electromagnetic field 258 over that which would be produced by a straight portion of signal conductor wire or a coil without the inductance-enhancing element 238. It should be appreciated that the magnetic portion of the field 258 will have a polarity dependant on the direction of the current flowing through the coil.

### VII. Signal Conduction Wire

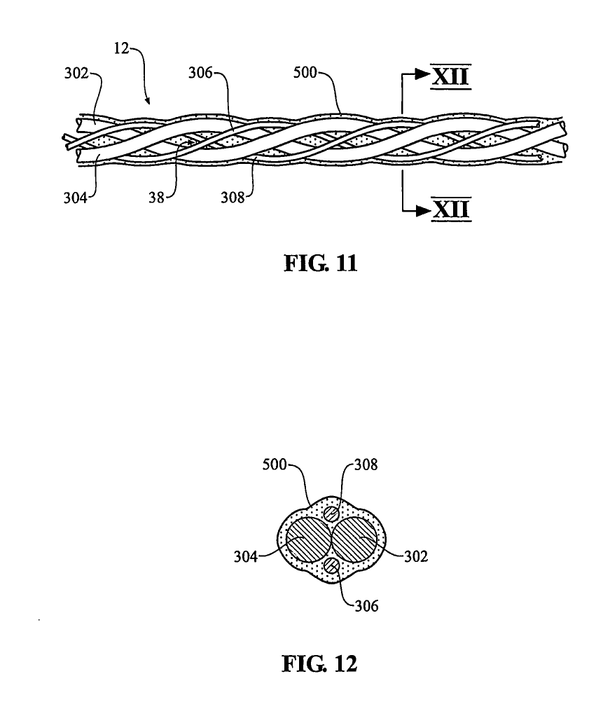

FIG. 11 depicts a portion of the guiding insert assembly 12 for a particular application wherein the elongated stiffener 39 includes a pair of twisted wires 302 and 304 and signal conduction wire assembly 38 wound thereon having a cover 500. The illustration shown in FIG. 11 is depicted with gaps between wires for clarity however the gaps between the wires would not exist in the actual windings and will have a spatial relationship more like that depicted in FIG. 12.

Since the signal conduction wire is a single wire, it is wound onto the elongated stiffener wire in a manner so as to finish up with the configuration shown in FIG. 11 where the conduction wire is wound about itself as well as the elongated stiffener. Although there are a number of ways of achieving the result depicted, the winding in one embodiment is achieved by first forming the electromagnetic field radiator 58 approximately in the middle of the signal conduction wire 202 and then winding one half of the signal conduction wire in one groove between the elongated stiffening wire and the other half of the signal conduction wire in the other groove along the length of the elongated stiffener up to a connector. Thus, the signal conductor includes a wire, the wire having a polymeric coating, ends of the wire being operatively coupled to a respective contact member of the connector 36, and the signal conductor wire being twisted about itself along its length (it not being necessary for the wire to be contacting itself to be twisted about itself, even though the signal conductor wire is twisted about a twisted stiffening wire assembly).

The twisting of the signal conduction wire in the manner described helps to minimize the radiation of electromagnetic fields from the elongated portion of the signal conduction wire assembly 38. Referring to FIGS. 11 and 12, since the electrical current being carried to the electromagnetic field radiator 58 is carried in one wire 306 that effectively runs parallel to the wire 308 and which carries current in the opposite direction the electromagnetic field generated by respective current flows substantially cancel each other out as will be understood by those skilled in the art.

Such an arrangement is useful in placing the electromagnetic fields generated by the current directly opposite to each other. FIG. 12 depicts a cross-section of the guiding insert assembly 12 at A-A showing the elongated stiffener wires 302 and 304 and the signal conduction wire segments 306/308 running parallel to each other.

FIG. 12 also shows a shape conforming cover 500 that may be provided by heat shrink material, a polyurethane or other shape conforming material that is non-conductive and useable in medical application areas.

### VIII. Guiding Insert Assemblies II and III

The types of guiding insert assembly described in detail thus far have been of the type including an elongate stiffener of the wire type, much akin to a stylet but which also carry a signal conductor wire and an electromagnetic field radiator. Another type of elongate stiffener is a tube, much like a catheter and in some embodiments made of the same material as a catheter.

When a tube like elongate stiffener is used the signal conductor wire and an electromagnetic field radiator are carried within the tube to become the guiding insert assembly. Its insertion into a catheter will stiffen the catheter making it easier for the health care provider to manipulate the catheter into position and when used in conjunction with the catheter position guidance system makes the task easier and safer for the patient.

The outer diameter of the elongate stiffener tube will determine the size of catheter it is most suited to be placed into during use, noting that some catheters have more than one lumen (continuous passage from their proximal end to their distal end for communicating fluids) and that the guiding insert assembly tube needs to be able to fit into and be extracted from the lumen without substantial frictional inteference. As the minimum inner diameter of the lumen may be as small as 0.05 mm the size of the tube must be the same or smaller in outer diameter. Furthermore, the outer diameter of the electromagnetic field radiator must be smaller again so as to fit within the guiding insert assembly tube when such an embodiment of the guiding insert assembly is created.

Furthermore, the type of material the tube is made of will also determine the stiffness of the tube, such as for example when the tube material is chosen from the polymer group of compounds it is likely to be stiffer than when the material is chosen from the silicon group of compounds. These two types of materials are not the only materials that can be used. Even if the tube material is the same as the catheter it is used within the use of a tube within a tube will tend to stiffen the catheter when being inserted into the patient.

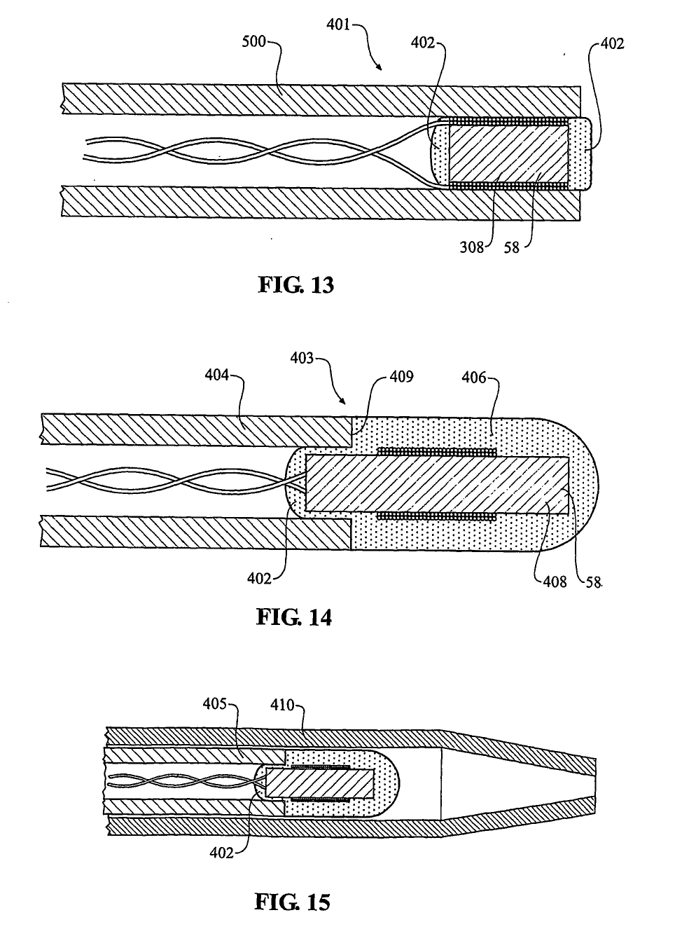

FIGS. 13 and 14 depict embodiments of tube like guiding insert assemblies. FIG. 13 shows a configuration of a guiding insert assembly 401 where the electromagnetic field radiator, 58 is located wholly within the tube 500 and plugged at its distal end (when in use) with non-conductive adhesive 402 or similar compound to seal off the end of the tube and fixing the location of the electromagnetic field radiator 58 adjacent the distal end of the tube 500. The electromagnetic field radiator 58 is constructed externally of the tube 500 and the signal conduction wire used to form the electromagnetic field radiator has either previously been passed through the tube 500 or is passed through the tube following formation of the electromagnetic field radiator. The electromagnetic field-radiator 58 and an application of non-conductive adhesive or similar compound is then pulled back or placed into the distal end of the tube 500 and becomes fixed in position adjacent the distal end of the tube 500.

The electromagnetic field radiator 58 may also, and will most likely, have when the radiator coil has a very small diameter, an inductance-enhancing material as a former for the coil.

The signal conducting wire is preferably twisted on itself to form an assembly that minimizes electromagnetic radiation from the elongated portion of the signal conductor wire.

FIG. 14 depicts a further embodiment of a tube like guiding insert assembly 403 wherein the electromagnetic field radiator 58 is located external of the tube 404. Such an arrangement allows the maximum outer diameter of the electromagnetic field radiator coil and covering material 406 to match closely the outer diameter of the tube 404. The cover material 406 may be a non-conductive adhesive or similar compound that fixes the end of the former 408 (which may be an inductance-enhancing material and which has equal and symmetric portions not covered by coil to that covered by the coil) in the distal end 409 of the tube 404 and encapsulates the otherwise exposed electromagnetic field radiator coil, in a smooth outer shape.

FIG. 15 depicts an illustration of the use of a tube like guiding insert assembly 405 (FIG. 14) inserted in a PICC line catheter 410.

Each of the guiding insert assemblies described can be sized to suit their application and in a common variation they are sized specifically for adult and pediatric use where smaller sizes (outer diameter as well as length) are required for pediatric application using catheters of smaller outer and inner dimensions as well as length to match the much smaller body and passages of children compared to an adult.

### IX. Use of the Guiding Insert Assembly with the Catheter Position Guidance System

In operation, the receiver 32 detects the electromagnetic field or signal 258 generated by the electromagnetic field radiator 58 inside the human body. The processor 20 causes the display device 22 and the hard copy device 43 to produce graphics 37 which assist the health care provider in the catheter placement procedure.

In one example, depicted by way of example only in FIG. 1, the system 2 is used by first determining the length of the catheter 50.

In some embodiments of the use of a guiding insert assembly the length of the catheter is exact and the corresponding length of the guiding insert assembly can be made to measure. Thus, the two items are supplied pre-matched with the guiding insert assembly suitable for the particular catheter already installed and ready to be connected directly to the apparatus 10 or to an intermediate connector possibly of the type described earlier in the specification and depicted in FIG. 7A.

In most catheters currently used, it will be necessary to match the length of the catheter to the patient and this is done by firstly estimating the length of catheter that is needed by measurements made on the surface of the patient and guidance from procedures laid down for such equipment usage. The catheter can be cut at its distal or proximal end to suit.

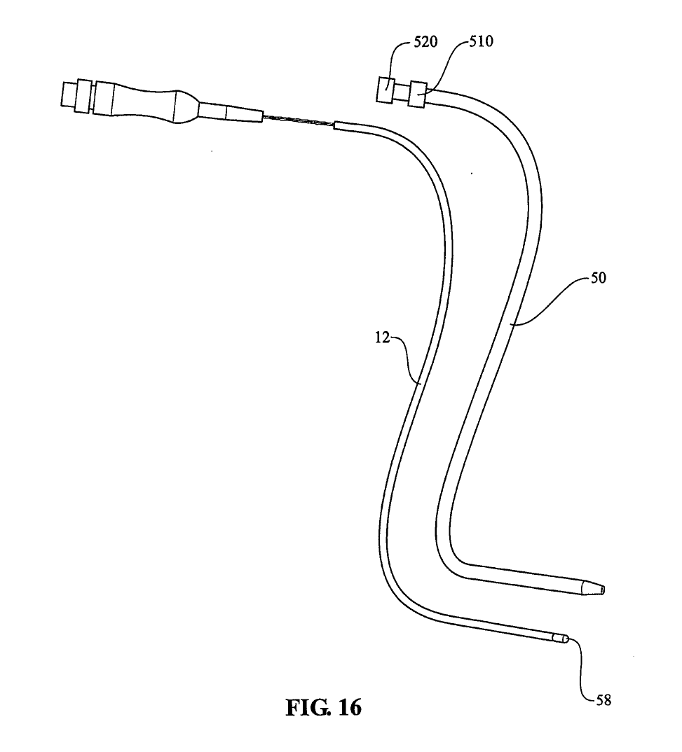

In some examples of the use of the guiding insert assembly, prior to placing the catheter 50 into the human body for enteral or parenteral feeding as shown in FIG. 16, the user or assembler places the guiding insert assembly 12 (in this example the guiding insert assembly has a tube elongated stiffener—see FIG. 14) having an electromagnetic field radiator 58 at its distal end into the catheter 50. In one arrangement the electromagnetic field radiator 58 is located adjacent the tip 60 of the catheter as is depicted in cross-sectional detail in FIG. 15.

It may be advantageous to fix the length of the guiding insert assembly 12 in the catheter and this can be done in one embodiment with a Leur lock connection 510 on the proximal end of the catheter connected to a Touhy Borst adaptor 520 through which the guiding insert passes and which can be locked relative to the catheter by the operation of the Touhy Borst adaptor.

The health care provider then places the receiver 32 on the patient's chest and inserts the catheter 50 into the body. Note, that the guiding insert assembly 16 is only required within the catheter during the placement of the catheter in the body of the patient. The guiding insert assembly is unlocked from the Touhy Borst and removed once placement has been achieved.

The catheter is then used to deliver, in an enteral example, nutrients to the required location in the stomach or other internal parts of the gastrointestinal tract. While for an application involving veins or arteries the catheter is used primarily, but without limitation, for the delivery of drugs to the required location in the body, often the cardio-vascular system of the body.

While the catheter is being located, the display device 22 displays graphics 37 that help the user in guiding the catheter tip 60 to a desired location within the human body. Those graphics are not the same as an X-ray and the graphical image is only a representation that can be useful only as an indication of the location of the distal end of the catheter to the health care provider. Once the catheter 50 is placed in the desired location, the user removes the guiding insert assembly 12 while the position of the catheter 50 is maintained. The tubular cover 40 can then be removed from the connector and the y-port if used and may then be disposed of.

The user can then, in an example, attach medicine and nutritional delivery tubes to an appropriate port of the y-port connector 44 for introducing fluids into the body for medical treatment. The use of y-port connectors is known for such feeding or delivery of drugs.

It would be understood that, in alternate embodiments, the guiding insert assembly of the present invention need not include a radiator position control device as the assemblers may measure each catheter and disregard each catheter that is too long or too short. It should be appreciated that other assembly processes and mechanisms may be used to control the proper location of the electromagnetic field radiator 58 relative to any portion of the catheter, in particular the tip 60.

It should also be appreciated that the assembly shown pictorially in FIG. 1, guiding insert assembly and catheter position guidance system of the present invention can be used in a variety of catheters and catheter procedures and applications. These procedures may involve the treatment of the gastrointestinal tract, cardiovascular system or other portions of the human body. These procedures may involve treatment of humans by physicians, clinicians, physician assistants, nurses or other health care providers. In addition, these procedures may involve treatment of other mammals and animals by veterinarians, researchers and others.

The present invention, in one embodiment, includes an electromagnetic field radiator for a guiding insert assembly of a catheter position guidance system. The electromagnetic field radiator is used in conjunction with other components of the system to assist the user in performing a catheter placement procedure. Also, the electromagnetic field radiator has an inductance-enhancing element, which enhances the performance of the system. Therefore, the electromagnetic field radiator, used in conjunction with a catheter position guidance system, provides an enhancement in medical treatment.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A guiding insert assembly for use with a catheter having a distal end for insertion into a body and a catheter position guidance system having a processor, the guiding insert assembly comprising:
an elongated stiffener for stiffening the catheter when inserted therein and having a first end and a distal end located, when inserted in the catheter, adjacent the distal end of the catheter;
a signal conductor wire connectable to the processor by its two ends, the signal conductor wire forming a winding along a portion of the length of the elongated stiffener and further forming an electromagnetic field radiator located adjacent the distal end of the elongated stiffener; wherein
the distal end of the elongated stiffener is non-conductively coupled to the electromagnetic field radiator.

2. The guiding insert assembly of claim 1, wherein the signal conductor wire includes a relatively thin coating.

3. The guiding insert assembly of claim 1, wherein the elongated stiffener forms a first segment and a second segment, the first and second segments being twisted around each other forming a twisted assembly at least a portion of the distance between the first end and the distal end of the elongated stiffener.

4. The guiding insert assembly of claim 1, which includes an inductance-enhancing element operatively associated with the electromagnetic field radiator.

5. The guiding insert assembly of claim 4, wherein the inductance-enhancing element has a ferromagnetic characteristic.

6. The guiding insert assembly of claim 5, wherein the inductance-enhancing element has an outer surface and an inner wall defining an opening, a portion of the signal conductor wire being wrapped around the outer surface to form a first layer of coil and a second layer of coil, the first and second layers of coils having a plurality of spirals, the spirals of the second layer of coil being uniformly patterned relative to the spirals of the first layer of coil.

7. The guiding insert assembly of claim 6, wherein the coupling between a portion of the second end of the elongated stiffener and the electromagnetic field radiator occurs in the opening of the inductance-enhancing element.

8. The guiding insert assembly of claim 4, wherein the inductance-enhancing element is a magnet.

9. The guiding insert assembly of claim 1, wherein the elongate stiffener is a tube.

10. The guiding insert assembly of claim 9, wherein the electromagnetic field radiator is located substantially external of the distal end of the tube.

11. A guiding insert assembly for use in conjunction with a catheter position guidance system having a processor, the guiding insert assembly comprising:
a connector having a top surface, a bottom surface, and at least one fastener attaching the top surface to the bottom surface, a plurality of contact members, said contact members operatively connectable to the processor;
a signal conductor includes a wire, the wire having a relatively thin coating, ends of the wire being operatively coupled to a respective contact member, the wire being twisted about itself along its length;
an elongated stiffener assembly having a first end and a second end, a first end of the elongated stiffener being coupled to a portion of the connector, and forming a first segment and a second segment, the first and second segments being twisted around each other, the signal conductor wire assembly being twisted about the elongated stiffener;
an inductance-enhancing element, an outer surface and an inner wall defining an opening, a portion of the signal conductor wire being wrapped around the outer surface to form at least one coil, the coil having a plurality of spirals;
a protectant encapsulating the inductance-enhancing element and the at least one coil; and
a coupling between a portion of the second end of the elongated stiffener and the inductance-enhancing element within the opening of the inductance-enhancing element.

12. The guiding insert assembly of claim 11, wherein the top and bottom surfaces of the connector are attached to each other by a mechanical or chemical fastener.

13. The guiding insert assembly of claim 12, wherein the mechanical fastener includes a fastener selected from the group consisting of a snap, screw and rivet.

14. The guiding insert assembly of claim 12, wherein the chemical fastener includes a fastener selected from the group consisting of an adhesive, chemical bond, weld bond and moulding.

15. The guiding insert assembly of claim 11, wherein the signal conductor wire includes a low resistance material.

16. The guiding insert assembly of claim 11, wherein the twisting of the signal conductor wire includes a range of 500 to 600 twists per metre along the lengths.

17. The guiding insert assembly of claim 11, wherein the elongated stiffener includes a ferrous material.

18. The guiding insert assembly of claim 11, wherein the elongate stiffener is a tube.

19. The guiding insert assembly of claim 18, wherein the electromagnetic field radiator is located substantially external of the distal end of the tube.

20. A guiding insert assembly for use in conjunction with a catheter position guidance system having a processor in communication with a display device and a receiver, the guiding insert assembly comprising:
a connector having a plurality of contact members for being operatively coupled to the processor;
a signal conductor including at least one wire coupled to the connector;
an elongated stiffener having a first end and a second end, said first end being coupled to the connector; and
an electromagnetic field radiator operatively formed at the distal end of the signal conductor and coupled to the second end of the elongated stiffener, the electromagnetic energy radiator operable to generate an electromagnetic field detectable by the receiver, the receiver communicating a signal to the processor based on said field, the processing causing the display device to display, and wherein the electromagnetic field radiator enables a graphical representation of catheter information based, at least in part, on said signal.

21. The guiding insert assembly of claim 20, wherein the signal conductor includes a wire having a relatively thin coating.

22. The guiding insert assembly of claim 20, wherein each wire of the signal conductor forms a twisted assembly along a portion between the connector and the second end of the elongate stiffener.

23. The guiding insert assembly of claim 20, wherein the elongated stiffener forms a first segment and a second segment, the first and second segments being twisted around each other, forming a twisted assembly, each signal conductor wire being twisted along a portion of the twisted assembly of the elongated stiffener.

24. The guiding insert assembly of claim 20, which includes an inductance-enhancing element operatively associated with the electromagnetic field radiator.

25. The guiding insert assembly of claim 24, wherein the inductance-enhancing element has a ferromagnetic characteristic.

26. The guiding insert assembly of claim 24, wherein the inductance-enhancing element has a ferromagnetic characteristic, an outer surface and an inner wall defining an opening, a portion of the signal conductor wire being wrapped around the outer surface to form a first layer of coil and a second layer of coil, the first and second layers of coils having a plurality of spirals, the spirals of the second layer of coil being uniformly patterned relative to the spirals of the first layer of coil.

27. The guiding insert assembly of claim 26, wherein the coupling between a portion of the second end of the elongated stiffener and the electromagnetic field radiator occurs in the opening of the inductance-enhancing element.

28. The guiding insert assembly of claim 20, wherein the elongate stiffener is a tube.

29. The guiding insert assembly of claim 28, wherein the electromagnetic field radiator is located substantially external of the distal end of the tube.

30. The guiding insert assembly of claim 20, wherein a signal conductor wire has a range of 500 to 600 twists per metre along its length.

31. A guiding insert assembly for use in conjunction with a catheter position guidance system having a processor, the guiding insert assembly including:
a signal conductor including at least one wire for being operatively coupled to the processor;
an elongated stiffener having a first end and a second end, said first end being fixed relative to the signal conductor;
an electromagnetic field radiator located adjacent the second end of the elongated stiffener and connected to the conductor; and
a coupling between a portion of the second end of the elongated stiffener and the electromagnetic field radiator.

32. A guiding insert assembly in accordance with claim 31 further comprising:
an inductance-enhancing element located in operative proximity to the electromagnetic field radiator.

33. A guiding insert assembly in accordance with claim 31, wherein the inductance-enhancing element has a ferromagnetic characteristics.

34. A guiding insert assembly in accordance with claim 31, wherein the inductance-enhancing element is a magnet.

35. A guiding insert assembly in accordance with claim 31, wherein the inductance-enhancing element has an outer surface and an inner wall defining an opening, a portion of the signal conductor wire being wrapped around the outer surface to form a first layer of coil and a second layer coil, the first and second coils having a plurality of spirals, the spirals of the second layer of coil being uniformly patterned relative to the spirals of the first layer of coil.

36. A guiding insert assembly in accordance with claim 35, wherein the coupling between a portion of the second end of the elongated stiffener and the electromagnetic field radiator occurs in the opening of the inductance-enhancing element.

37. A guiding insert assembly in accordance with claim 31, wherein the conductor includes a relatively thin coating.

38. A guiding insert assembly in accordance with claim 31, wherein the conductor is a twisted wire a portion of the distance between the processor and the electromagnetic field radiator.

39. The guiding insert assembly of claim 31, wherein the elongate stiffener is a tube.

40. The guiding insert assembly of claim 39, wherein the electromagnetic field radiator is located substantially external of the distal end of the tube.

41-50. (canceled)
